(19) United States
(12) Patent Application Publication
(10) Pub. No.: US 2025/0391515 A1
(43) Pub. Date: Dec. 25, 2025

(54) DETERMINING PHENOMIC RELATIONSHIPS BETWEEN COMPOUNDS AND CELL PERTURBATIONS UTILIZING MACHINE LEARNING MODELS

(71) Applicant: Recursion Pharmaceuticals, Inc., Salt Lake City, UT (US)

(72) Inventors: Oscar MENDEZ LUCIO, Madrid (ES); Stephen Scott MACKINNON, Burlington (CA); Christodoulos Antoniou NIKOLAOU, Carmel, IN (US)

(21) Appl. No.: 18/753,906

(22) Filed: Jun. 25, 2024

(51) Int. Cl. G16C 20/30; G16C 20/70

(52) U.S. Cl. CPC G16C 20/30; G16C 20/70

(57) ABSTRACT

The present disclosure relates to systems, non-transitory computer-readable media, and methods for training and utilizing machine learning models to generate structure-phenomics relationship predictions for cell perturbations. In particular, in some embodiments, the disclosed systems receive a query chemical compound. In addition, in some embodiments, the disclosed systems generate a compound structure feature representation for the query chemical compound. Moreover, in some embodiments, the disclosed systems generate, utilizing a structure-phenomics relationship machine learning model, a phenomic similarity prediction for the compound structure feature representation and a target perturbation.

### BACKGROUND

Recent years have seen developments in hardware and software platforms for training and utilizing machine learning models for generating predictions. For example, existing systems utilize large volumes of training data to teach machine learning models to generate intelligent predictions corresponding to complex biological interactions between genes, compounds, and/or proteins. Despite these recent developments, existing systems suffer from a number of technical deficiencies, particularly with regard to accuracy, efficiency, and operational flexibility in implementing machine learning technologies.

### BRIEF SUMMARY

Embodiments of the present disclosure provide benefits and/or solve one or more problems in the art with systems, non-transitory computer-readable media, and methods for determining phenomic relationships (e.g., impacts within a cell) between query compounds and cell perturbations utilizing one or more machine learning models. In some embodiments, the disclosed systems utilize a machine learning model to analyze a structural feature representation of a compound to predict a phenomic relationship between the compound and one or more cell perturbations (e.g., treatment perturbations, such as other chemical compounds or gene knockout sequences). For example, the disclosed systems can determine a structural feature representation for the chemical compound and generate a phenomic similarity prediction from the compound structural feature representation and a treatment perturbation. Moreover, in some embodiments, the disclosed systems utilize phenomic similarity predictions in drug discovery pipelines or other downstream tasks.

The following description sets forth additional features and advantages of one or more embodiments of the disclosed methods, non-transitory computer-readable media, and systems. In some cases, such features and advantages are evident to a skilled artisan having the benefit of this disclosure, or may be learned by the practice of the disclosed embodiments.

### BRIEF DESCRIPTION OF THE DRAWINGS

The detailed description provides one or more embodiments with additional specificity and detail through the use of the accompanying drawings, as briefly described below.

FIG. 1 illustrates an overview of a structure-phenomics relationship (sphere) system in accordance with one or more embodiments.

FIG. 2 illustrates the sphere system generating phenomic data from compounds and perturbations in accordance with one or more embodiments.

FIGS. 3A-3B illustrate the sphere system determining gene-specific upper and lower pheno-similarity thresholds from distributions of difference metrics for two different gene perturbations in accordance with one or more embodiments.

FIG. 4 illustrates the sphere system utilizing a training compound to learn parameters of a structure-phenomics relationship machine learning model in accordance with one or more embodiments.

FIGS. 5A-5B illustrate the sphere system providing a graphical user interface for receiving a query compound and providing a phenomic similarity prediction for display in accordance with one or more embodiments.

FIG. 6 illustrates a diagram of an environment in which a structure-phenomics relationship system operates in accordance with one or more embodiments.

FIG. 7 illustrates a flowchart of a series of acts for generating a phenomic similarity prediction in accordance with one or more embodiments.

FIG. 8 illustrates a block diagram of an example computing device for implementing one or more embodiments of the present disclosure.

### DETAILED DESCRIPTION

This disclosure describes one or more embodiments of a structure-phenomics relationship (“sphere”) system that utilizes machine learning models to predict phenomic relationships between chemical compounds and cell perturbations (e.g., gene knockout sequences or other chemical compounds). For example, the sphere system can determine a structural feature representation for the chemical compound and generate a phenomic similarity prediction (e.g., a classification prediction for similarity in cell phenotype resulting from application of the chemical compound relative to one or more perturbations). Moreover, in some embodiments, the sphere system utilizes the phenomic similarity predictions in a drug discovery pipeline or other task. For example, the sphere system can utilize phenomic similarity predictions to select compounds for subsequent testing, to confirm structure-phenomic relationships with genes for mechanism of action analysis, to find chemical compounds with promising pheno-similarity characteristics, and/or to supplement a phenomap and actively update the machine learning model for enhanced phenomic similarity predictions.

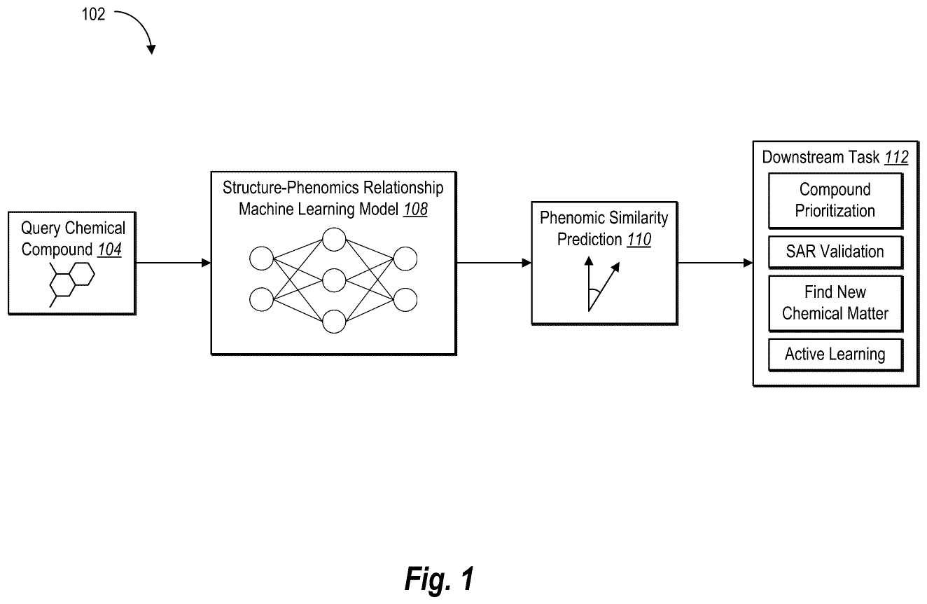

As mentioned, in some embodiments, the sphere system generates phenomic similarity predictions for query compounds relative to gene perturbations (or other cell perturbations). As shown in FIG. 1, a sphere system 102 receives a query chemical compound 104. For instance, in some implementations, the query chemical compound 104 is an input chemical structure or a chemical compound selected from a library of compounds.

In some embodiments, the sphere system 102 utilizes a structure-phenomics relationship machine learning model 108 to process the query chemical compound 104. For instance, the sphere system 102 utilizes the structure-phenomics relationship machine learning model 108 to generate a phenomic similarity prediction 110 for the query chemical compound 104 and one or more perturbations. As discussed with additional detail below, in some implementations, the phenomic similarity prediction 110 includes a score or a classification that denotes a phenomic similarity between the query chemical compound 104 and a perturbation. For example, the phenomic similarity prediction 110 denotes a predicted similarity of the bioactivity of the query chemical compound 104 (as applied to a living cell) to the bioactivity of the perturbation (as applied to the living cell). Thus, the phenomic similarity prediction 110 can indicate a similarity classification indicating a level of similarity in cell development, impact, or expression between a query compound and one or more other perturbations.

Moreover, in some embodiments, the sphere system 102 generates a compound structure feature representation for the query chemical compound 104. For instance, the sphere system 102 utilizes the structure-phenomics relationship machine learning model 108 or another machine learning model to generate a structure feature representation for the query chemical compound 104 and process the compound structure feature representation through the structure-phenomics relationship machine learning model 108 to generate the phenomic similarity prediction 110.

A structure feature representation includes a digital representation of a compound and its structural features (e.g., atoms, bonds, charges, and/or other chemical characteristics). In some implementations, the structure feature representation comprises a numerical representation of features of a chemical compound or a gene perturbation. For instance, a structure feature representation includes a vector representation of a chemical structure or a gene sequence. To illustrate, a structure feature representation includes a latent feature vector representation of a compound or gene generated by one or more layers of a neural network. For example, in some embodiments, the sphere system 102 generates a structure feature representation by processing a query chemical compound or a perturbation through one or more layers of a neural network (e.g., the structure-phenomics relationship machine learning model 108). In one or more implementations, a structure feature representation includes a graph representation (e.g., generated by a graph neural network, where edges represent bonds and nodes represent atoms of a compound). The structure feature representation can include (or be generated from) other digital feature representations of a compound, such as Simplified Molecular Input Line Entry System (SMILES), SMILES Arbitrary Target Specification (SMARTS), International Chemical Identifier (InChI), InChIKey, Molecular 2D/3D File Format (MOL2), Protein Data Bank Format (PDB), RDKit, XYZ Files, Canonical SMILES, or Tensor Representations, among others.

A machine learning model includes a computer representation that is tunable (e.g., trained) based on inputs to approximate unknown functions used for generating corresponding outputs. In particular, in one or more embodiments, a machine learning model is a computer-implemented model that utilizes algorithms to learn from, and make predictions on, known data by analyzing the known data to learn to generate outputs that reflect patterns and attributes of the known data. For instance, in some cases, a machine learning model includes, but is not limited to, a neural network (e.g., a convolutional neural network, recurrent neural network, or other deep learning network), a decision tree (e.g., a gradient boosted decision tree), support vector learning, Bayesian networks, a transformer-based model, a diffusion model, or a combination thereof.

Similarly, a neural network includes a machine learning model that is trainable and/or tunable based on inputs to determine classifications and/or scores, or to approximate unknown functions. For example, in some cases, a neural network includes a model of interconnected artificial neurons (e.g., organized in layers) that communicate and learn to approximate complex functions and generate outputs based on inputs provided to the neural network. In some cases, a neural network refers to an algorithm (or set of algorithms) that implements deep learning techniques to model high-level abstractions in data. A neural network includes various layers such as an input layer, one or more hidden layers, and an output layer that each perform tasks for processing data. For example, a neural network includes a deep neural network, a convolutional neural network, a diffusion neural network, a recurrent neural network (e.g., an LSTM), a graph neural network, a transformer, or a generative adversarial neural network.

In some embodiments, the sphere system 102 utilizes a transformer-based model as the structure-phenomics relationship machine learning model 108 that analyzes a graph representation of an input compound. For example, in some embodiments, the sphere system 102 utilizes one or more of the models described by Méndez-Lucio et al. in MolE: A Molecular Foundation Model for Drug Discovery, arXiv.2211.02657, November 2022, which is incorporated by reference herein in its entirety.

In some implementations, the sphere system 102 utilizes a multi-task model (e.g., a model with multiple task heads) as the structure-phenomics relationship machine learning model 108, whereby the sphere system 102 can generate phenomic similarity predictions for a plurality of (e.g., numerous) gene perturbations or other perturbations for the query chemical compound 104. To illustrate, the sphere system 102 can train classification task heads (e.g., neural network prediction heads) for different gene perturbations. The sphere system 102 can then utilize the task heads to generate predictions for the gene perturbations from a structural feature representation of an input compound. For instance, the sphere system 102 can utilize the compound structure feature representation for the query chemical compound 104 to generate phenomic similarity predictions for genes in a library of thousands of genes.

As mentioned, in some embodiments, the sphere system 102 utilizes the phenomic similarity prediction 110 in a downstream task 112. For example, the sphere system 102 utilizes the phenomic similarity prediction 110 for compound prioritization, SAR validation, finding new chemical matter, and/or active learning.

To illustrate, in compound prioritization, the sphere system 102 utilizes phenomic similarity predictions to select compounds to test in one or more programs (e.g., ICG expansion or lead optimization). Moreover, in SAR validation, the sphere system 102 confirms structure-phenomic relationships with genes for mechanism of action analysis and/or target deconvolution. In addition, the sphere system 102 can find new chemical matter by searching compound libraries for compounds with promising pheno-similarity characteristics (e.g., relative to the query chemical compound 104). Furthermore, in active learning, the sphere system 102 can identify compounds to test and add to the phenomap (e.g., a repository of phenomic data including pheno-similarity classifications) or that will be most informative to the structure-phenomics relationship machine learning model 108.

As mentioned previously, conventional systems have a number of technical problems with regard to efficiency, accuracy, and operational flexibility of implementing computing devices. For example, in order to determine phenomic similarity of compounds or genes, conventional systems often engage in complex procedures that require extensive time and computational resources. For example, conventional systems can perform a variety of cell assays and generate machine learning embeddings that represent one or more cell modifications. Conventional systems can compare these embeddings to determine similarities between compounds and/or genes. These processes, however, require significant processing power, memory, and time to determine phenomic similarities.

Without such extensive testing, conventional systems struggle to identify phenomic similarities, thus undermining the accuracy and operational flexibility of conventional systems. Indeed, as just mentioned, conventional systems are tied to a rigid approach for determining phenomic similarities. This makes conventional systems unable to rapidly and flexibly analyze new compounds to determine phenomic relationships.

The sphere system 102 provides a variety of technical advantages relative to existing systems. For example, the sphere system 102 improves efficiency relative to conventional systems. Indeed, the sphere system 102 can generate phenomic similarity predictions by analyzing structural features of input compounds. Accordingly, once trained, the sphere system 102 can utilize a structure-phenomics relationship machine learning model to generate phenomic similarity predictions (e.g., to identify other genes or compounds that result in similar cell bioactivity) while avoiding the time and resources needed by conventional systems to execute robotic assays, generate and store machine learning embeddings, and compare such embeddings. Thus, the sphere system 102 reduces time, processing power, and memory required to analyze novel compounds and generate phenomic similarity predictions.

Furthermore, the sphere system 102 provides improved accuracy and operational flexibility by determining pheno-similarity between chemical compounds and genes (without the need for complex processes of conventional systems). Indeed, when presented with a new compound, the sphere system 102 can directly analyze structural features of the new compound and predict phenomic similarity of the new compound relative to various gene/compound perturbations. Accordingly, the sphere system 102 can accurately identify genes and/or compounds with similar cellular impacts by flexibly analyzing the compound features themselves. Specifically, the sphere system 102 provides a novel pipeline for training and utilizing machine learning models to generate predictions of phenomic relationships between various cell perturbations. For example, by training the structure-phenomics relationship machine learning model 108 with phenomic similarity classifications generated from phenomic image embeddings (as described below), the sphere system 102 provides new capabilities for accurately predicting phenomic similarity without requiring extensive laboratory testing.

Moreover, the sphere system 102 can provide enhanced accuracy of biosimilarity predictions by tailoring the comparisons of cell perturbations to individual genes. For example, and as described in additional detail below, the sphere system 102 determines gene-specific pheno-similarity thresholds for each gene. The sphere system 102 utilizes the gene-specific pheno-similarity thresholds to provide additional accuracy improvements in determining phenomic similarity predictions between query chemical compounds and treatment perturbations.

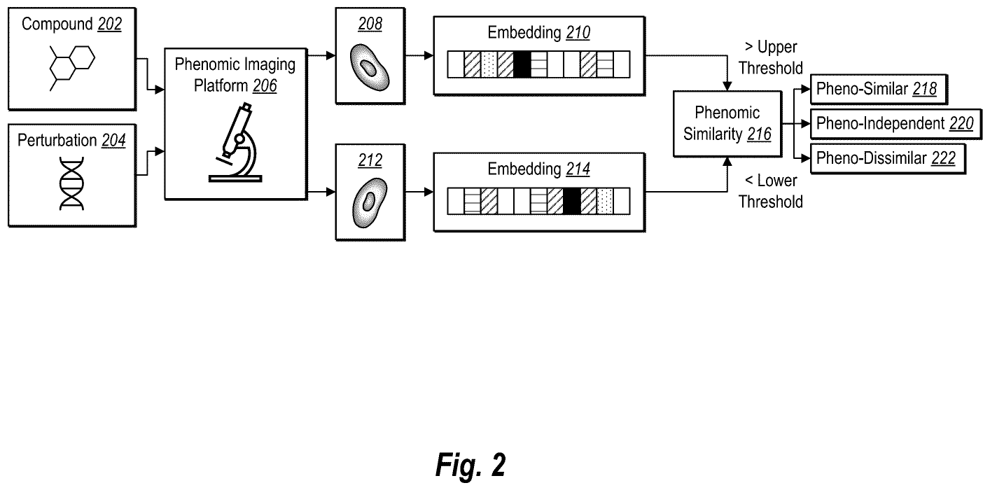

As discussed, in some embodiments, the sphere system 102 leverages phenomic data to generate phenomic similarity predictions. For instance, FIG. 2 illustrates the sphere system 102 generating phenomic data from compounds and perturbations in accordance with one or more embodiments.

Specifically, FIG. 2 shows the sphere system 102 obtaining a compound 202. For example, the sphere system 102 receives a chemical compound to apply to a biological cell (e.g., a human cell, an animal cell, etc.). Additionally, the sphere system 102 identifies/applies a perturbation 204 (e.g., applies the perturbation 204 to a cell). For instance, the sphere system 102 utilizes a gene knockout on a similar biological cell. Upon exposure to the compound 202 or the perturbation 204, a cell may undergo a change (e.g., a physical or biological change) expressed as a phenotypic change.

In some implementations, the sphere system 102 utilizes a phenomic imaging platform 206 to capture images (e.g., digital images) of the cells exposed to the compound 202 and the perturbation 204, respectively. In particular, the sphere system 102 utilizes a camera to capture a first phenomic image 208 of a first cell exposed to the compound 202, and a second phenomic image 212 of a second cell exposed to the perturbation 204.

Moreover, in some embodiments, the sphere system 102 generates embeddings of the phenomic images. For instance, the sphere system 102 generates a first embedding 210 of the first phenomic image 208 and a second embedding 214 of the second phenomic image 212. To illustrate, the sphere system 102 performs (e.g., utilizing robotic assay implementation devices) cell perturbations and captures phenomic digital images of the perturbed cells. Specifically, the sphere system 102 performs a machine learning analysis on the digital images portraying perturbed cells to generate embeddings from the phenomic digital images and compares the embeddings to identify inter-relationships between genes, proteins, compounds, and/or diseases. Thus, the sphere system 102 generates a phenomic similarity prediction from phenomic embeddings of a machine learning model generated from digital images portraying cells exposed to various perturbations.

To illustrate, in some implementations, the sphere system 102 generates phenomic embeddings as described in U.S. patent application Ser. No. 18/392,989, titled UTILIZING MACHINE LEARNING AND DIGITAL EMBEDDING PROCESSES TO GENERATE DIGITAL MAPS OF BIOLOGY AND USER INTERFACES FOR EVALUATING MAP EFFICACY, filed on Dec. 21, 2023 (hereinafter '989 Patent), which is incorporated by reference herein in its entirety. Additionally, in some cases, the sphere system 102 can utilize a machine learning model trained to generate predicted cell representations from masked cell representations as described in U.S. patent application Ser. No. 18/545,399, titled UTILIZING MASKED AUTOENCODER GENERATIVE MODELS TO EXTRACT MICROSCOPY REPRESENTATION AUTOENCODER EMBEDDINGS, filed on Dec. 19, 2023, which is incorporated by reference herein in its entirety.

In some implementations, the sphere system 102 compares the first embedding 210 and the second embedding 214 to determine a phenomic similarity 216. For example, the sphere system 102 determines a similarity score (e.g., utilizing a cosine similarity or other similarity metric such as a distance metric or projection metric) of the embeddings. To illustrate, the similarity score is a numerical metric representing commonalties (or lack thereof) in bioactivity between the compound 202 and the perturbation 204 as expressed in their phenomic data.

Furthermore, in some implementations, the sphere system 102 utilizes thresholds to classify the similarity score. In some embodiments, the sphere system 102 classifies pairs of compounds and perturbations utilizing one of three classifications: a pheno-similar classification 218 (e.g., for compounds and perturbations that share common bioactivity); a pheno-independent classification 220 (e.g., for compounds and perturbations that have orthogonal bioactivity); or a pheno-dissimilar classification 222 (e.g., for compounds and perturbations that have opposite bioactivity). For example, if the similarity score exceeds an upper threshold, the sphere system 102 classifies the phenomic similarity as pheno-similar; if the similarity score is less than a lower threshold, the sphere system 102 classifies the phenomic similarity as pheno-dissimilar; and if the similarity score falls between the upper and lower thresholds, the sphere system 102 classifies the phenomic similarity as pheno-independent. In some implementations, the sphere system 102 utilizes a different number of classifications (e.g., two classifications, such as dependent or independent).

Although the description herein often refers to single cells, it will be appreciated that the sphere system 102 can apply perturbations and generate embeddings for a plurality of cells (e.g., a population of cells). Thus, the sphere system 102 can apply a first perturbation to a plurality of cells, develop the plurality of cells, and capture a plurality of images. Moreover, the sphere system 102 can generate a plurality of cell representation embeddings. In some implementations, the sphere system 102 generates a cell representation embedding from a plurality of cells (e.g., by combining cell representations from a plurality of cells to form a cell embedding for a particular perturbation). Thus, for example, the sphere system 102 can generate a first cell embedding by aggregating a plurality of cell representation embeddings from a plurality of cells exposed to a first perturbation. Similarly, the sphere system 102 can generate a second cell representation embedding by aggregating a plurality of cell representation embeddings from a plurality of cells exposed to a second perturbation. In some implementations, the sphere system 102 utilizes the process described in the '989 Patent.

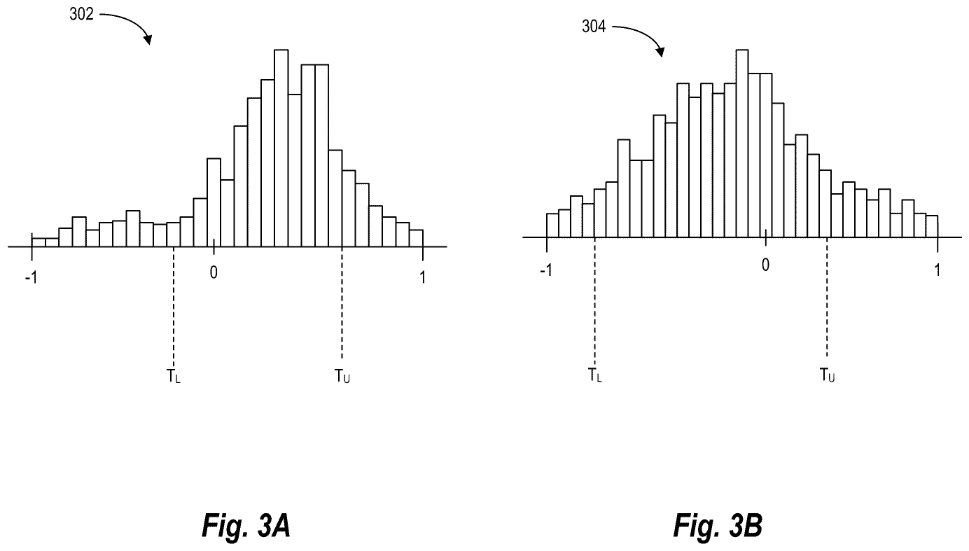

As just mentioned, in some embodiments, the sphere system 102 utilizes upper and lower thresholds to classify phenomic similarities. In particular, in some implementations, the sphere system 102 utilizes a gene-specific upper threshold and a gene-specific lower threshold to classify phenomic similarities for a particular gene perturbation. For instance, FIGS. 3A and 3B illustrate the sphere system 102 determining different upper and lower thresholds for different gene perturbations in accordance with one or more embodiments. Specifically, FIG. 3A shows a first distribution 302 of difference metrics (e.g., pheno-similarity scores) for a first perturbation, while FIG. 3B shows a second distribution 304 of difference metrics for a second perturbation.

In some implementations, the sphere system 102 generates difference metrics between a perturbation (e.g., a gene knockout) and numerous other perturbations (e.g., numerous chemical compounds). For example, the sphere system 102 generates a distribution of difference metrics between the perturbation and a plurality of additional perturbations. Moreover, in some implementations, the sphere system 102 generates difference metrics between the perturbation and multiple different concentrations of an additional perturbation (e.g., a chemical compound). To illustrate, the sphere system 102 determines a first difference metric between a gene and a first concentration of a first compound, a second difference metric between the gene and a second concentration of the first compound, a third difference metric between the gene and a first concentration of a second compound, and a fourth difference metric between the gene and a second concentration of the second compound. Similarly, in some cases, the sphere system 102 generates many (e.g., hundreds) difference metrics between a gene and many different concentrations of each of numerous (e.g., thousands) compounds to compile a distribution of difference metrics that has a multitude (e.g., millions) of metrics for the single gene.

Thus, the first distribution 302 of difference metrics can have a first multitude of pheno-similarity scores for a first gene and the second distribution 304 of difference metrics can have a second multitude of pheno-similarity scores for a second gene. As the various distributions of difference metrics are different for each gene perturbation, the sphere system 102 determines unique thresholds for each gene perturbation. By way of illustration, and not limitation, the sphere system 102 can assign a lower threshold for the first distribution 302 of around −0.3 and an upper threshold for the first distribution 302 of around 0.6. In contrast, by way of illustration, and not limitation, the sphere system 102 can assign a lower threshold for the second distribution 304 of around −0.8 and an upper threshold for the second distribution 304 of around 0.4.

In some embodiments, the sphere system 102 utilizes one or more of a variety of statistical tools to determine the upper and lower thresholds of pheno-similarity scores for a particular perturbation. For example, the sphere system 102 considers whether the distribution is symmetrical, whether the distribution is Gaussian, and whether the distribution has long tails on either side. The sphere system 102 can apply different thresholds to different distributions. In some implementations, the sphere system 102 utilizes an interquartile range, a semi-interquartile range, and/or a median absolute deviation.

By determining pheno-similarity thresholds that are unique to particular genes, the sphere system 102 can enhance the accuracy of phenomic similarity predictions with respect to those genes because different genes have different phenotypic behaviors. For example, some genes have stronger phenotypes than others, and therefore generally produce higher pheno-similarity scores than others. Thus, the sphere system 102 tailors the pheno-similarity thresholds to reflect gene-specific relationships and map those relationships into the training data for the structure-phenomics relationship machine learning model 108.

In some implementations, the sphere system 102 determines that a compound is pheno-similar to a gene perturbation if at least one concentration of the compound has a phenomic similarity above the upper pheno-similarity threshold. For example, if the maximum phenomic similarity score for the pairing of the compound with the gene exceeds the upper threshold for that gene, the sphere system 102 classifies the compound as pheno-similar to the gene. In alternative implementations, the sphere system 102 utilizes an average (e.g., mean) or a minimum of the phenomic similarity scores for the several concentrations of the compound with respect to the gene.

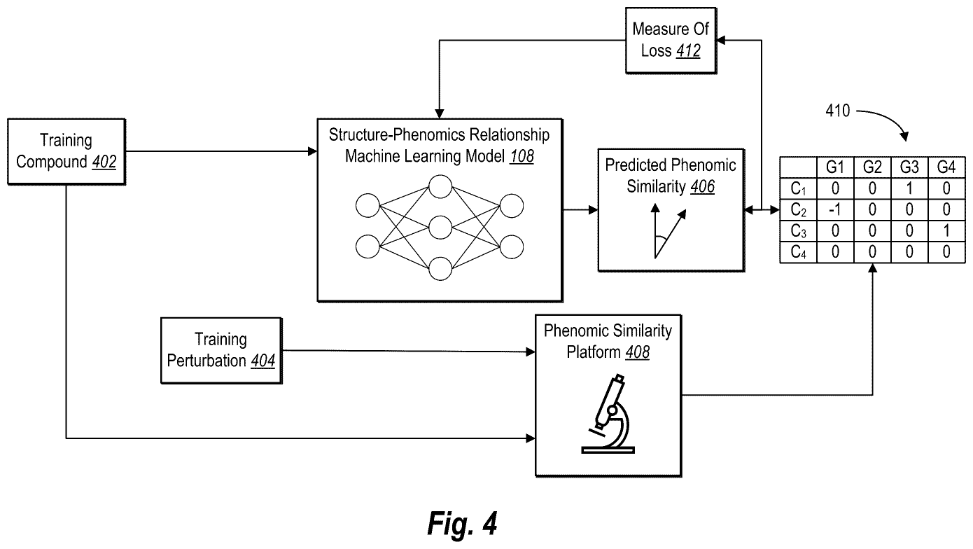

As mentioned, in some embodiments, the sphere system 102 trains a structure-phenomics relationship machine learning model to generate phenomic similarity predictions. For instance, FIG. 4 illustrates the sphere system 102 utilizing a training compound and a training perturbation to learn parameters of the structure-phenomics relationship machine learning model in accordance with one or more embodiments.

Specifically, FIG. 4 shows the sphere system 102 obtaining a training compound 402 and a training perturbation 404. The sphere system 102 processes the training compound 402 through the structure-phenomics relationship machine learning model 108 to generate a predicted phenomic similarity 406. Additionally, the sphere system 102 utilizes a phenomic similarity platform 408 to generate a similarity matrix 410 of phenomic image similarities from training compounds and training perturbations.

As just mentioned, in some embodiments, the sphere system 102 utilizes the phenomic similarity platform 408 to generate the similarity matrix 410. For instance, the sphere system 102 utilizes the phenomic similarity platform 408 to perform the techniques described above in connection with FIG. 2. In particular, the sphere system 102 utilizes the phenomic similarity platform 408 to capture phenomic images of cells exposed, respectively, to the training compound 402 and the training perturbation 404. Additionally, the sphere system 102 utilizes the phenomic similarity platform 408 to identify machine learning embeddings corresponding to the training compound 402 and the training perturbation 404. For example, the sphere system 102 identifies a first machine learning embedding of a first phenomic image of a first cell exposed to the training compound 402, and a second machine learning embedding of a second phenomic image of a second cell exposed to the training perturbation 404.

Moreover, the sphere system 102 compares the first machine learning embedding and the second machine learning embedding to generate a phenomic image similarity for the training compound 402 and the training perturbation 404. For instance, the sphere system 102 generates a difference metric (e.g., a cosine similarity or other similarity metric) between the first machine learning embedding and the second machine learning embedding. Additionally, the sphere system 102 applies a pheno-similarity threshold to the difference metric to generate a pheno-similarity classification (e.g., pheno-similar, pheno-dissimilar, or pheno-independent) between the training compound 402 and the training perturbation 404. For example, the sphere system 102 applies a pheno-similarity threshold unique to the training perturbation 404. For instance, the sphere system 102 determines the pheno-similarity threshold from a distribution of difference metrics between the training perturbation 404 and a plurality of additional perturbations (e.g., additional compounds), as described above in connection with FIGS. 3A and 3B.

In some embodiments, the sphere system 102 adds the pheno-similarity classification between the training compound 402 and the training perturbation 404 to the similarity matrix 410. For example, the sphere system 102 builds the similarity matrix 410 to include a table of pheno-similarity classifications for numerous training compounds (e.g., as rows of the table) and numerous training perturbations (e.g., as columns of the table). Although described as a matrix, the sphere system 102 can collect pheno-similarity classifications in a variety of different digital representations, including a matrix, array, or table.

As mentioned, in some implementations, the sphere system 102 utilizes the structure-phenomics relationship machine learning model 108 to generate the predicted phenomic similarity 406. To illustrate, in some embodiments, the sphere system 102 generates a training compound structure feature representation for the training compound 402. The sphere system 102 utilizes the structure-phenomics relationship machine learning model 108 to generate the predicted phenomic similarity 406 from the training compound structure feature representation.

The sphere system 102 can generate the predicted phenomic similarity 406 utilizing the training compound 402 in a variety of approaches. As mentioned previously, in some implementations, the sphere system 102 trains a variety of different task heads for different perturbations. For example, the sphere system 102 can select a task head specific to the training perturbation 404 for a training iteration. The sphere system 102 can then process the training compound 402 utilizing the structure-phenomics relationship machine learning model 108 and the specific task head to generate the predicted phenomic similarity 406 between the training compound 402 and the training perturbation.

In some implementations, the sphere system 102 has a plurality of training heads for a plurality of perturbations and generates a predicted phenomic similarity for each perturbation of the plurality of perturbations. The sphere system 102 then trains each task head by comparing the predicted phenomic similarity with the corresponding measured (ground truth) similarity for that particular compound-perturbation pair. In this manner, the sphere system 102 can train the structure-phenomics relationship machine learning model 108 to generate similarity predictions for a plurality of different perturbations in a consolidated training approach. Once trained, the structure-phenomics relationship machine learning model 108 can generate phenomic similarity predictions for the plurality of perturbations from any given input compound.

As mentioned, in some embodiments, the sphere system 102 modifies parameters of the structure-phenomics relationship machine learning model 108 to train the structure-phenomics relationship machine learning model 108 to predict phenomic similarities between query compounds and any number of perturbations. To illustrate, the sphere system 102 compares the predicted phenomic similarity 406 with the phenomic image similarity (e.g., the pheno-similarity classification in the similarity matrix 410 corresponding to the training compound 402 and the training perturbation 404) to determine a measure of loss 412. For instance, the sphere system 102 determines a difference between the predicted phenomic similarity 406 and the phenomic image similarity. Based on the measure of loss 412, the sphere system 102 modifies parameters of the structure-phenomics relationship machine learning model 108 (e.g., to improve the structure-phenomics relationship machine learning model 108 by reducing measures of loss of subsequent iterations of training). For example, the sphere system 102 can utilize back-propagation and/or gradient descent techniques to modify parameters of the structure-phenomics relationship machine learning model 108 to reduce the measure of loss. The sphere system 102 can iteratively perform such training processes (e.g., utilizing different training batches) to train the structure-phenomics relationship machine learning model 108 (e.g., until reaching a threshold number of training iterations or until reaching a threshold accuracy/measure of loss).

Upon training the structure-phenomics relationship machine learning model 108, the sphere system 102 can utilize the structure-phenomics relationship machine learning model 108 to analyze structural features of input compounds (e.g., the query chemical compound 104 described above in connection with FIG. 1) and generate phenomic similarity predictions for the input compounds with respect to other perturbations (e.g., other treatment perturbations). For example, the sphere system 102 can predict whether a query compound will be pheno-similar to, pheno-independent of, or pheno-dissimilar to one or more genes (or other perturbations, such as other chemical compounds). In some implementations, the sphere system 102 can generate other predictions (e.g., different classifications such as dependent or independent, or numerical similarity predictions).

In some alternative embodiments, the sphere system 102 trains the structure-phenomics relationship machine learning model 108 utilizing other types of biological data than phenomic images and phenomic image embeddings. For example, in some implementations, the sphere system 102 utilizes RNA data applicable to a gene perturbation. For example, the sphere system 102 determines biosimilarity predictions for a compound or gene perturbation based on RNA counts associated with the gene perturbation. To illustrate, the sphere system 102 can perform perturbation assays and (instead of capturing digital images), utilize sequencing machines to determine a number of transcription proteins (e.g., mRNA) resulting from the perturbation. The sphere system 102 can build a transcriptomic profile for a perturbation that reflects the number of transcription proteins resulting from a particular perturbation. The sphere system 102 can also build a transcriptomic matrix indicating transcriptomic profiles across a plurality of perturbations. Further, the sphere system 102 can compare transcriptomic profiles of different perturbations to determine transcriptomic similarity scores.

In some implementations, the sphere system 102 utilizes these transcriptomic similarity scores (rather than phenomic similarity measures) to train a machine learning model. Thus, the sphere system 102 can utilize a machine learning model to predict transcriptomic similarity. In addition to phenomic and transcriptomic similarity, the sphere system 102 can also train a machine learning model to generate other-omics predictions (e.g., inivomics predictions reflecting liability predictions or reactions of animals exposed to a particular perturbation).

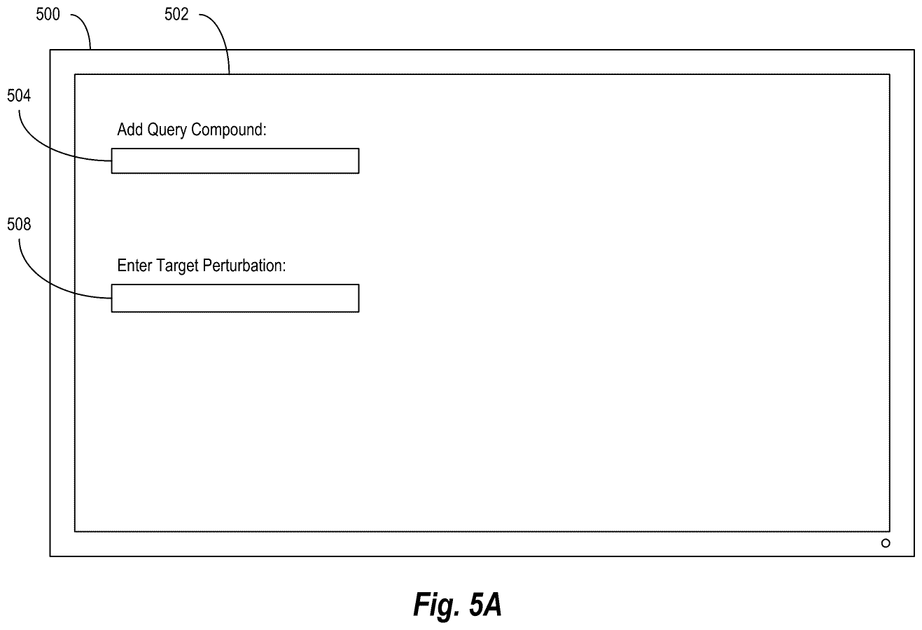

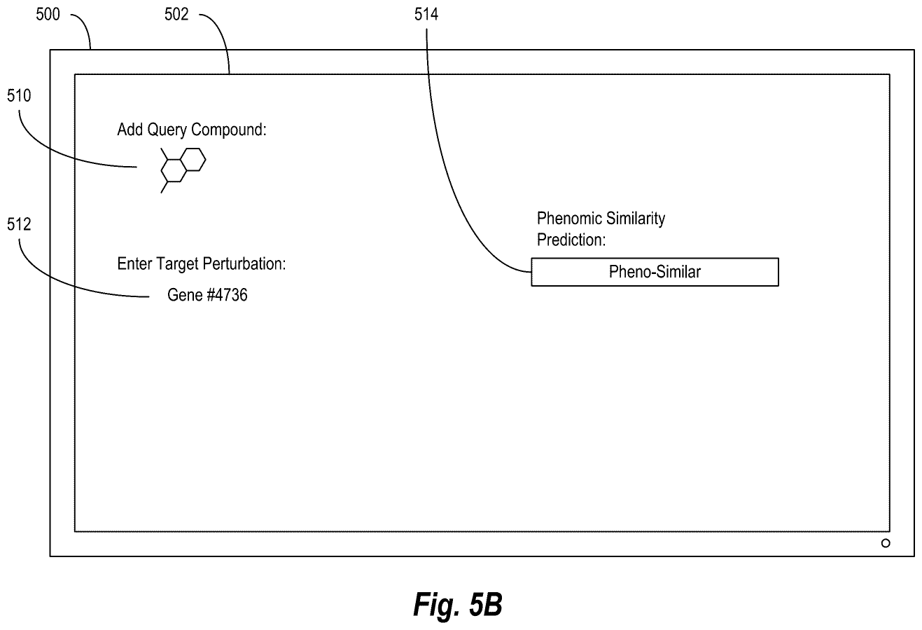

As mentioned, in some embodiments, the sphere system 102 provides a user interface via which a user may interact with the sphere system 102. For instance, FIGS. 5A and 5B illustrate the sphere system 102 providing a graphical user interface for display via a computing device in accordance with one or more embodiments.

Specifically, FIG. 5A shows a client device 500 with a graphical user interface 502. In some implementations, the sphere system 102 provides the graphical user interface 502 for display via the client device 500. The graphical user interface 502 includes an input element 504 for a query chemical compound and an input element 508 for a target perturbation. Thus, a user can enter a query compound (e.g., the query chemical compound 104) via the input element 504. Similarly, a user can enter a target perturbation (e.g., another chemical compound or another treatment, such as a gene perturbation) via the input element 508. Alternatively, in some embodiments, the sphere system 102 runs the phenomic similarity techniques described herein on the query compound against a library (e.g., an array) of target perturbations. Thus, in some embodiments, a user inputs a query compound without also entering a target perturbation.

FIG. 5B shows the sphere system 102 receiving a query chemical compound 510 and a target perturbation 512 via the graphical user interface 502 of the client device 500. Moreover, FIG. 5B shows the sphere system 102 providing a phenomic similarity prediction 514 for display via the graphical user interface 502. For example, the sphere system 102 utilizes the phenomic similarity techniques described herein to determine the phenomic similarity prediction 514 for the query chemical compound 510 with respect to the target perturbation 512, and then provides the phenomic similarity prediction 514 for display.

As mentioned previously, in some embodiments, the sphere system 102 utilizes the phenomic similarity prediction in a variety of downstream tasks (e.g., in addition to providing the phenomic similarity prediction for display via a client device).

For example, in some implementations, the sphere system 102 can utilize phenomic similarity predictions for compound prioritization and/or SAR validation in one or more compound exploration programs. To illustrate, the sphere system 102 can include industrial program generation (IPG) and industrialized compound generation (ICG). For instance, industrial program generation (IPG) includes (i) a hit selection to identify statistically strong connections in a biological map (e.g., phenomic of phenomic embeddings) to patient-informed phenotypes, (ii) phenomic confirmation (e.g., promising actives are confirmed by automated similarity and concentration-response analytics), (iii) Trekseq confirmation (e.g., compound and gene relationships are confirmed with transcriptomics in the map background), and (iv) Structure-Activity Relationship (SAR) confidence (e.g., analysis of the relationship between the chemical structure of compounds and their biological activity)).

ICG applies to steps subsequent to IPG. Further, in some embodiments ICG includes rapidly searching and expanding from potential hit series in the chemical space (e.g., identified at the IPG stage) and testing the potential hits with various analytical tests (e.g., SAR screens). The sphere system 102 can utilize phenomic similarity predictions to inform a variety of these downstream tasks within a compound exploration program. Indeed, the sphere system 102 can utilize phenomic similarity predictions in deciding to initiate one of these compound exploration programs, in performing hit selection, in performing phenomic confirmation or Trekseq confirmation, in SAR confidence (e.g., analyzing behavior/bioactivity of compound variations by analyzing similarities to other compounds/genes), in expanding compounds (and determining how those compound variations behave), and performing SAR screens.

In addition, the sphere system 102 can find new chemical matter by searching compound libraries for compounds. In particular, the sphere system 102 can generate structural feature representations of a variety of novel compounds and generate similarity predictions relative to a variety of different perturbations. The sphere system 102 can thus identify compounds within the compound libraries that have similar predicted phenotypic behavior of previously explored genes/compounds. This can assist in selecting new compounds for additional program analysis.

Furthermore, in active learning, the sphere system 102 can identify promising compounds for performing actual assays and generating additional machine learning embeddings. Indeed, as mentioned above, performing assays and generating phenomic image embeddings is a time consuming and computationally expensive task. The sphere system 102 can further improve efficiency by utilizing phenomic similarity predictions to identify the most promising compounds for these tasks. Thus, the sphere system 102 can utilize a structure-phenomics relationship machine learning model to generate predicted phenomic similarities for a variety of compounds and then select a subset of those compounds for actual phenomic image embeddings based on the predicted phenomic similarities (e.g., those compounds that appear to be most promising for further exploration). These embeddings can then be utilized for additional training of a structure-phenomics relationship machine learning model or other machine learning models. In this manner, the sphere system 102 can utilize predicted phenomic similarities for active learning and identification of targets for additional testing and active improvement of existing machine learning processes.

The sphere system 102 can also utilize the structure-phenomic relationship machine learning model as part of a generative process for creating a new/modified compound in response to a query. Indeed, the structure-phenomic relationship machine learning model can be implemented as part of a larger generative model (e.g., graph neural network) that learns the structural properties of molecules for generating new compounds. For instance, the structure-phenomic relationship machine learning model can be used as part of an oracle for guiding a generative model (e.g., a graph neural network or other generative model such as a generative adversarial neural network, variational autoencoder, reinforcement learning model, autoregressive model, transformer such as large language model, diffusion model, or flow based model). For example, the structure-phenomic relationship machine learning model can act as an oracle to evaluate generated compounds (e.g., chemical properties, biological interactions) and provide feedback to guide the model in generating new or novel compounds as part of a larger graph neural network.

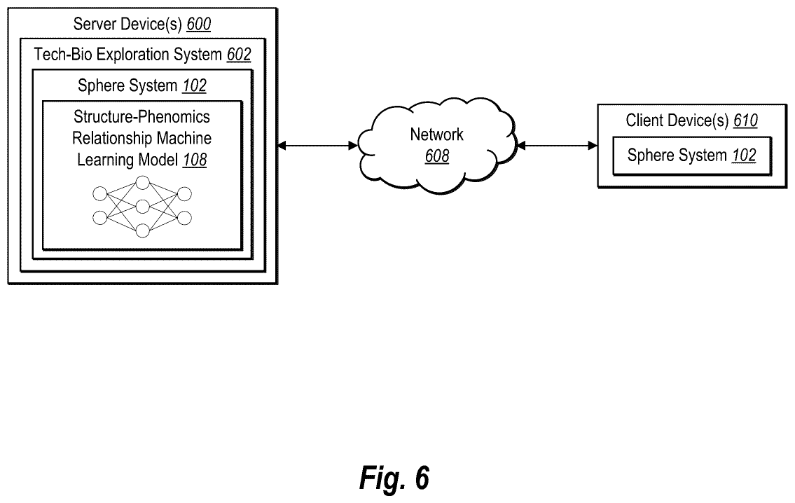

Additional detail regarding the computing environment in which the sphere system 102 operates will now be provided with reference to FIG. 6. In particular, FIG. 6 illustrates a schematic diagram of a system environment in which the sphere system 102 can operate in accordance with one or more embodiments.

As shown in FIG. 6, the environment includes server device(s) 600 (which includes a tech-bio exploration system 602 and the sphere system 102), client device(s) 610, and a network 608. As further illustrated in FIG. 6, the various computing devices within the environment can communicate via the network 608. Although FIG. 6 illustrates the sphere system 102 being implemented by a particular component and/or device within the environment, the sphere system 102 can be implemented—in whole or in part—by other computing devices and/or components in the environment (e.g., additional client device(s)). Additional description regarding the illustrated computing devices is provided with respect to FIG. 8 below.

As shown in FIG. 6, the server device(s) 600 (e.g., one or more local servers operated by a particular entity) can include the tech-bio exploration system 602. In some embodiments, the tech-bio exploration system 602 can determine, store, generate, and/or provide for display tech-bio information including maps of biology, experiments from various sources, and/or machine learning tech-bio predictions. For instance, the tech-bio exploration system 602 can analyze data signals corresponding to various treatments or interventions (e.g., compounds or biologics) and the corresponding relationships in genetics, proteomics, phenomics (e.g., cellular phenotypes), and invivomics (e.g., expressions or results within a living animal). Moreover, the tech-bio exploration system 602 provides an environment for operating, executing, and/or managing complex drug discovery pipelines.

For instance, the tech-bio exploration system 602 can generate and access experimental results corresponding to gene sequences, protein shapes/folding, protein/compound interactions, phenotypes resulting from various interventions or perturbations (e.g., gene knockout sequences or compound treatments), and/or in vivo experimentation on various treatments in living animals. By analyzing these signals (e.g., utilizing various machine learning models), the tech-bio exploration system 602 can generate or determine a variety of predictions and inter-relationships for improving treatments/interventions.

To illustrate, the tech-bio exploration system 602 can generate maps of biology indicating biological inter-relationships or similarities between these various input signals to discover potential new treatments as part of the complex compound discovery process. For example, the tech-bio exploration system 602 can utilize machine learning and/or maps of biology to identify a similarity between a first gene associated with disease treatment and a second gene previously unassociated with the disease based on a similarity in resulting phenotypes from gene knockout experiments. The tech-bio exploration system 602 can then identify new treatments based on the gene similarity (e.g., by targeting compounds the impact the second gene). Similarly, the tech-bio exploration system 602 can analyze signals from a variety of sources (e.g., protein interactions, in vivo experiments) to predict efficacious treatments based on various levels of biological data.

The tech-bio exploration system 602 can generate GUIs comprising dynamic user interface elements to convey tech-bio information and receive user input for intelligently exploring tech-bio information. Indeed, as mentioned above, the tech-bio exploration system 602 can generate GUIs displaying different maps of biology that intuitively and efficiently express complex interactions between different biological systems for identifying improved treatment solutions. Furthermore, the tech-bio exploration system 602 can also electronically communicate tech-bio information between various computing devices.

The tech-bio exploration system 602 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 602 collects, manages, and transmits data across a variety of different entities, accounts, and devices. In some cases, the tech-bio exploration system 602 is a network system that facilitates access to (and analysis of) tech-bio information within a centralized operating system. Indeed, the tech-bio exploration system 602 can link data from different network-based research institutions to generate and analyze maps of biology.

As shown in FIG. 6, the tech-bio exploration system 602 can include the sphere system 102 that generates, stores, manages, and transmits data pertaining to phenomic similarities. For example, in the context of the above description for the tech-bio exploration system 602, in some embodiments, the tech-bio exploration system 602 further utilizes the sphere system 102 to enhance the coordination between various groups involved in the drug discovery process. For instance, the sphere system 102 works in tandem with the tech-bio exploration system 602 to generate phenomic similarity predictions, transmit the phenomic similarity predictions to one or more devices, and initiate one or more downstream model predictions or processes.

As also illustrated in FIG. 6, the environment includes the client device(s) 610. As mentioned above, the client device(s) 610 can be involved in the process of drug discovery. Thus, for example, the client device(s) 610 can coordinate/manage a first stage of generating a phenomic similarity prediction (e.g., for a protein and a compound). Moreover, the client device(s) 610 can coordinate/manage a second stage such as generating a bioactivity prediction based on the phenomic similarity prediction. Further, the client device(s) 610 can coordinate/manage a third stage of utilizing the bioactivity prediction to generate one or more additional predictions or initiate one or more programs (IPG or ICG).

To illustrate, the client device(s) 610 can include computing devices that implement or manage a compound program generation stage of a compound discovery process. Similarly, the client device(s) 610 can include computing devices that implement or manage a compound lead generation stage and the client device(s) 610 can include computing devices that implement or manage a compound/dose selection stage. For example, the sphere system 102 can receive one or more requests to utilize the structure-phenomics relationship machine learning model 108 to generate one or more phenomic similarity predictions. For instance, the sphere system 102 can receive additional requests from the client device(s) 610 that include generating the bioactivity predictions.

In some embodiments, the environment also includes additional device(s). For example, the sphere system 102 can utilize the additional device(s) to further operate and manage the completion of complex drug discovery pipelines. For instance, the additional device(s) include experimental device(s) and analytical device(s). Further, in some instances, the additional device(s) also include the computing devices discussed below in connection with FIG. 8.

Furthermore, in one or more implementations, the client device(s) 610 include a client application. The client application can include instructions that (upon execution) cause the client device(s) 610 to perform various actions. For example, a user of a user account can interact with the client application on the client device(s) 610 to execute experiments or other multi-faceted processes, to further access tech-bio information, and/or initiate a request for a phenomic similarity prediction. For instance, in some embodiments the sphere system 102 receives a request to generate a phenomic similarity prediction, and in response generates the prediction and returns the prediction to the client device(s) 610. In some instances, the transmittal of the phenomic similarity prediction to the client device(s) 610 causes the client device(s) 610 to execute an action (e.g., generate a downstream model prediction or other task).

Additionally, the environment can include dedicated machine learning device(s). For example, the dedicated machine learning device(s) can include computing devices or virtual machines dedicated to training or implementing large-scale machine learning models. For example, the dedicated machine learning device(s) can generate machine learning predictions and/or embeddings based on digital biological data (e.g., digital images of phenotypes resulting from different perturbations or compound-protein interactions from compound features). Thus, the sphere system 102 can interact with the dedicated machine learning device(s) to generate the phenomic similarity prediction.

The environment can also include experimental device(s). For example, the tech-bio exploration system 602 can interact with experimental device(s) that include intelligent robotic devices and camera devices for generating and capturing digital images of cellular phenotypes resulting from different perturbations (e.g., genetic knockouts or compound treatments of stem cells). Similarly, the experimental device(s) can include camera devices and/or other sensors (e.g., heat or motion sensors) capturing real-time information from animals as part of in vivo experimentation. The tech-bio exploration system 602 can also interact with a variety of other experimental device(s) such as devices for determining, generating, or extracting gene sequences or protein information. For example, the experimental device(s) may include computing devices linked to biosensorselectrophysiological platforms, x-ray crystallography machines, liquid chromatography mass spectrometry systems, nuclear magnetic resonance spectrometers, and/or mass spectrometers. In some implementations, the sphere system 102 generates the phenomic similarity predictions and further determines to employ or utilize one or more experimental devices (e.g., to initiate one or more experiments based on the phenomic similarity predictions).

As further shown in FIG. 6, the environment includes the network 608. As mentioned above, the network 608 can enable communication between components of the environment. In one or more embodiments, the network 608 may include a suitable network and may communicate using a various number of communication platforms and technologies suitable for transmitting data and/or communication signals, examples of which are described with reference to FIG. 8. Furthermore, although FIG. 6 illustrates computing devices communicating via the network 608, the various components of the environment can communicate and/or interact via other methods (e.g., communicate directly).

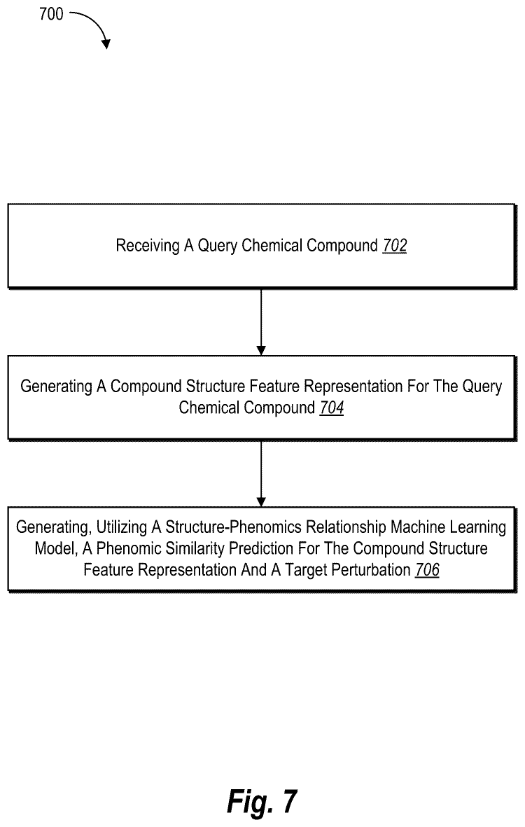

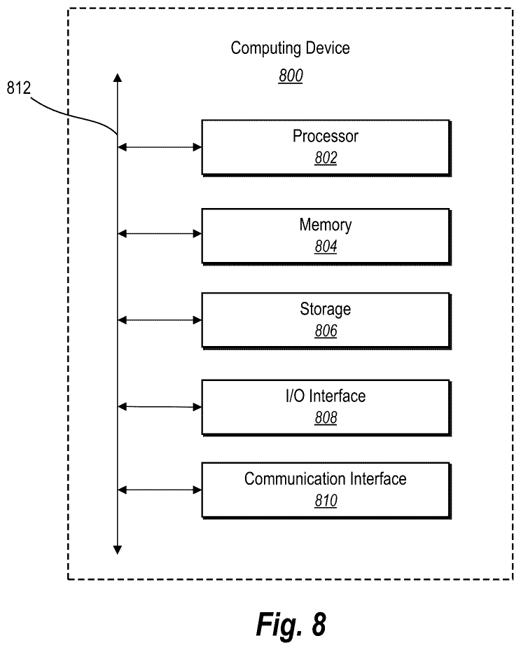

FIGS. 1-6, the corresponding text, and the examples provide a number of different methods, systems, devices, and non-transitory computer-readable media of the sphere system 102. In addition to the foregoing, one or more embodiments are described in terms of flowcharts comprising acts for accomplishing a particular result, as shown in FIG. 7. In some implementations, the processes of the sphere system 102 are performed with more or fewer acts. Furthermore, in various implementations, the acts are performed in differing orders. Additionally, in some implementations, the acts described herein are repeated or performed in parallel with one another or in parallel with different instances of the same or similar acts.

As mentioned, FIG. 7 illustrates a flowchart of a series of acts 700 for generating phenomic similarity predictions in accordance with one or more implementations. While FIG. 7 illustrates acts according to one implementation, alternative implementations omit, add to, reorder, and/or modify any of the acts shown in FIG. 7. In one or more implementations, the acts of FIG. 7 are performed as part of a method (e.g., a computer-implemented method). Alternatively, in one or more implementations, a non-transitory computer-readable storage medium comprises instructions that, when executed by one or more processors, cause a computing device to perform the acts of FIG. 7. In some implementations, a system performs the acts of FIG. 7.

As shown in FIG. 7, the series of acts 700 includes an act 702 of receiving a query chemical compound (and, some implementations, a target perturbation), an act 704 of generating a compound structure feature representation for the query chemical compound, and an act 706 of generating, utilizing a structure-phenomics relationship machine learning model, a phenomic similarity prediction for the compound structure feature representation and a target perturbation.

In particular, in some implementations, the act 702 includes receiving a query chemical compound, the act 704 includes generating a compound structure feature representation for the query chemical compound, and the act 706 includes generating, utilizing a structure-phenomics relationship machine learning model, a phenomic similarity prediction for the compound structure feature representation and a target perturbation, wherein the structure-phenomics relationship machine learning model is trained to generate phenomic similarity predictions utilizing training compound structure feature representations and phenomic image similarities generated by comparing phenomic images of cells exposed to cell perturbations.

For example, in some implementations, the series of acts 700 includes receiving the target perturbation by receiving a target gene knockout perturbation or a target compound perturbation. In addition, in some implementations, the series of acts 700 includes generating the phenomic similarity prediction by generating a similarity classification from a set of classifications comprising: a pheno-similar classification, a pheno-dissimilar classification, and a pheno-independent classification.

Moreover, in some implementations, the series of acts 700 includes training the structure-phenomics relationship machine learning model by: identifying a first machine learning embedding of a first phenomic image of a first cell exposed to a training compound; identifying a second machine learning embedding of a second phenomic image of a second cell exposed to a training perturbation; and generating a phenomic image similarity by comparing the first machine learning embedding and the second machine learning embedding.

For example, in some implementations, the series of acts 700 includes generating the phenomic image similarity by: generating a difference metric between the first machine learning embedding and the second machine learning embedding; and applying a pheno-similarity threshold to the difference metric to generate a pheno-similarity classification between the training compound and the training perturbation. Additionally, in some implementations, the series of acts 700 includes determining the pheno-similarity threshold from a distribution of difference metrics between the training perturbation and a plurality of additional perturbations.

Moreover, in some implementations, the series of acts 700 includes training the structure-phenomics relationship machine learning model by: generating a training compound structure feature representation for the training compound; and generating, utilizing the structure-phenomics relationship machine learning model, a predicted phenomic similarity from the training compound structure feature representation and the training perturbation. Furthermore, in some implementations, the series of acts 700 includes training the structure-phenomics relationship machine learning model by: comparing the predicted phenomic similarity with the phenomic image similarity to determine a measure of loss; and modifying parameters of the structure-phenomics relationship machine learning model based on the measure of loss.

In addition, in some implementations, the series of acts 700 includes receiving the query chemical compound and the target perturbation via a user interface of a client device; and providing the phenomic similarity prediction for display via the user interface of the client device.

Embodiments of the present disclosure may comprise or utilize a special purpose or general purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions from a non-transitory computer-readable medium (e.g., memory) and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or generators and/or other electronic devices. When information is transferred, or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface generator (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general purpose computer, special purpose computer, or special purpose processing device to perform a certain function or group of functions. In some embodiments, computer-executable instructions are executed by a general purpose computer to turn the general purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program generators may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), a web service, Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 8 illustrates a block diagram of an example computing device 800 that may be configured to perform one or more of the processes described above. One will appreciate that one or more computing devices, such as the computing device 800, may represent the computing devices described above (e.g., the server device(s) 600 or the client device(s) 610). In one or more embodiments, the computing device 800 may be a mobile device (e.g., a mobile telephone, a smartphone, a PDA, a tablet, a laptop, a camera, a tracker, a watch, a wearable device, etc.). In some embodiments, the computing device 800 may be a non-mobile device (e.g., a desktop computer or another type of client device). Further, the computing device 800 may be a server device that includes cloud-based processing and storage capabilities.

As shown in FIG. 8, the computing device 800 can include one or more processor(s) 802, memory 804, a storage device 806, input/output interfaces 808 (or “I/O interfaces 808”), and a communication interface 810, which may be communicatively coupled by way of a communication infrastructure (e.g., bus 812). While the computing device 800 is shown in FIG. 8, the components illustrated in FIG. 8 are not intended to be limiting. Additional or alternative components may be used in other embodiments. Furthermore, in certain embodiments, the computing device 800 includes fewer components than those shown in FIG. 8. Components of the computing device 800 shown in FIG. 8 will now be described in additional detail.

In particular embodiments, the processor(s) 802 includes hardware for executing instructions, such as those making up a computer program. As an example, and not by way of limitation, to execute instructions, the processor(s) 802 may retrieve (or fetch) the instructions from an internal register, an internal cache, memory 804, or a storage device 806 and decode and execute them.

The computing device 800 includes the memory 804, which is coupled to the processor(s) 802. The memory 804 may be used for storing data, metadata, and programs for execution by the processor(s). The memory 804 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 804 may be internal or distributed memory.

The computing device 800 includes the storage device 806 for storing data or instructions. As an example, and not by way of limitation, the storage device 806 can include a non-transitory storage medium described above. The storage device 806 may include a hard disk drive (“HDD”), flash memory, a Universal Serial Bus (“USB”) drive or a combination these or other storage devices.

As shown, the computing device 800 includes one or more I/O interfaces 808, which are provided to allow a user to provide input to (such as user strokes), receive output from, and otherwise transfer data to and from the computing device 800. These I/O interfaces 808 may include a mouse, keypad or a keyboard, a touch screen, camera, optical scanner, network interface, modem, other known I/O devices or a combination of such I/O interfaces 808. The touch screen may be activated with a stylus or a finger.

The I/O interfaces 808 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 808 are configured to provide graphical data to a display for presentation to a user. The graphical data may be representative of one or more graphical user interfaces and/or any other graphical content as may serve a particular implementation.

The computing device 800 can further include a communication interface 810. The communication interface 810 can include hardware, software, or both. The communication interface 810 provides one or more interfaces for communication (such as, for example, packet-based communication) between the computing device and one or more other computing devices or one or more networks. As an example, and not by way of limitation, communication interface 810 may include a network interface controller (“NIC”) or network adapter for communicating with an Ethernet or other wire-based network or a wireless NIC (“WNIC”) or wireless adapter for communicating with a wireless network, such as a WI-FI. The computing device 800 can further include the bus 812. The bus 812 can include hardware, software, or both that connects components of computing device 800 to each other.

The components of the sphere system 102 include software, hardware, or both. For example, the components include one or more instructions stored on a computer-readable storage medium and executable by processors of one or more computing devices, such as a client device or server device. When executed by the one or more processors, in some implementations, the computer-executable instructions of the sphere system 102 cause the computing device(s) to perform the methods described herein. Alternatively, in one or more implementations, the components include hardware, such as a special purpose processing device to perform a certain function or group of functions. Alternatively, in some implementations, the components of the sphere system 102 include a combination of computer-executable instructions and hardware.

Furthermore, the components of the sphere system 102 are, for example, implemented as one or more operating systems, as one or more stand-alone applications, as one or more modules of an application, as one or more plug-ins, as one or more library functions, as one or more functions callable by other applications, and/or as a cloud-computing model. Thus, in some implementations, the components are implemented as a stand-alone application, such as a desktop or mobile application. Furthermore, in various implementations, the components are implemented as one or more web-based applications hosted on a remote server. In some implementations, the components are implemented in a suite of mobile device applications or “apps.”

The use in the foregoing description and in the appended claims of the terms “first,” “second,” “third,” etc., is not necessarily to connote a specific order or number of elements. Generally, the terms “first,” “second,” “third,” etc., are used to distinguish between different elements as generic identifiers. Absent a showing that the terms “first,” “second,” “third,” etc., connote a specific order, these terms should not be understood to connote a specific order. Furthermore, absent a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget, and not necessarily to connote that the second widget has two sides.

In the foregoing description, the invention has been described with reference to specific exemplary embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with fewer or more steps/acts or the steps/acts may be performed in differing orders. Additionally, the steps/acts described herein may be repeated or performed in parallel with one another or in parallel with different instances of the same or similar steps/acts. The scope of the invention is, therefore, indicated by the appended claims rather than by the foregoing description. All changes that come within the meaning and range of equivalency of the claims are to be embraced within their scope.

1. A computer-implemented method comprising:
training a structure-phenomics relationship neural network to generate phenomic similarity predictions by:
generating, utilizing a trained embedding neural network, a first phenomic embedding from a first phenomic image of a first cell exposed to a training compound;
generating, utilizing the trained embedding neural network, a second phenomic embedding from a second phenomic image of a second cell exposed to a training perturbation;
comparing, within a latent feature space of the trained embedding neural network, the first phenomic embedding and the second phenomic embedding to generate a phenomic image feature space similarity;
generating, utilizing the structure-phenomics relationship neural network, a predicted phenomic feature space similarity between the training compound and the training perturbation from a training compound structure feature representation of the training compound;
comparing the phenomic image feature space similarity and the predicted phenomic feature space similarity to generate a phenomic feature space similarity measure of loss; and
modifying parameters of the structure-phenomics relationship neural network utilizing the phenomic feature space similarity measure of loss;
generating a compound structure feature representation for a query chemical compound; and
generating, utilizing the structure-phenomics relationship neural network, a phenomic similarity prediction for the compound structure feature representation and a target perturbation.

2. The computer-implemented method of claim 1, wherein the target perturbation comprises a target gene knockout perturbation or a target compound perturbation.

3. The computer-implemented method of claim 1, wherein generating the phenomic similarity prediction comprises generating a similarity classification from a set of classifications comprising: a pheno-similar classification, a pheno-dissimilar classification, and a pheno-independent classification.

4. The computer-implemented method of claim 1, wherein training the structure-phenomics relationship neural network further comprises modifying the parameters of the structure-phenomics relationship neural network to reduce the phenomic feature space similarity measure of loss on a subsequent training iteration.

5. The computer-implemented method of claim 1, further comprising generating the phenomic image feature space similarity by:
generating a difference metric between the first phenomic embedding and the second phenomic embedding; and
applying a pheno-similarity threshold to the difference metric to generate a pheno-similarity classification between the training compound and the training perturbation.

6. The computer-implemented method of claim 5, further comprising determining the pheno-similarity threshold from a distribution of difference metrics between the training perturbation and a plurality of additional perturbations.

7. The computer-implemented method of claim 1, wherein training the structure-phenomics relationship neural network further comprises generating, utilizing the structure-phenomics relationship neural network, an additional predicted phenomic feature space similarity from the training compound structure feature representation and an additional training perturbation.

8. The computer-implemented method of claim 7, wherein training the structure-phenomics relationship neural network further comprises:
modifying parameters of a first task head of the structure-phenomics relationship neural network based on the predicted phenomic feature space similarity corresponding to the training perturbation; and
modifying parameters of a second task head of the structure-phenomics relationship neural network based on the additional predicted phenomic feature space similarity corresponding to the additional training perturbation.

9. The computer-implemented method of claim 1, further comprising:
receiving the query chemical compound and the target perturbation via a user interface of a client device; and
providing the phenomic similarity prediction for display via the user interface of the client device.

10. A system comprising:
at least one processor; and
at least one non-transitory computer-readable storage medium storing instructions that, when executed by the at least one processor, cause the system to:
train a structure-phenomics relationship neural network to generate phenomic similarity predictions by:
generating, utilizing a trained embedding neural network, a first phenomic embedding from a first phenomic image of a first cell exposed to a training compound;
generating, utilizing the trained embedding neural network, a second phenomic embedding from a second phenomic image of a second cell exposed to a training perturbation;
comparing, within a latent feature space of the trained embedding neural network, the first phenomic embedding and the second phenomic embedding to generate a phenomic image feature space similarity;
generating, utilizing the structure-phenomics relationship neural network, a predicted phenomic feature space similarity between the training compound and the training perturbation from a training compound structure feature representation of the training compound;
comparing the phenomic image feature space similarity and the predicted phenomic feature space similarity to generate a phenomic feature space similarity measure of loss; and
modifying parameters of the structure-phenomics relationship neural network utilizing the phenomic feature space similarity measure of loss;
generate a compound structure feature representation for a query chemical compound; and
generate, utilizing the structure-phenomics relationship neural network, a phenomic similarity prediction for the compound structure feature representation and a target perturbation.

11. The system of claim 10, wherein the at least one non-transitory computer-readable storage medium stores additional instructions that, when executed by the at least one processor, cause the system to:
receive the target perturbation by receiving a target gene knockout perturbation or a target compound perturbation; and
generate the phenomic similarity prediction by generating a similarity classification from a set of classifications comprising: a pheno-similar classification, a pheno-dissimilar classification, and a pheno-independent classification.

12. The system of claim 10, wherein the at least one non-transitory computer-readable storage medium stores additional instructions that, when executed by the at least one processor, cause the system to train the structure-phenomics relationship neural network by:
modifying parameters of a first task head of the structure-phenomics relationship neural network based on the predicted phenomic feature space similarity corresponding to the training perturbation; and
modifying parameters of a second task head of the structure-phenomics relationship neural network based on an additional predicted phenomic feature space similarity corresponding to an additional training perturbation.

13. The system of claim 10, wherein the at least one non-transitory computer-readable storage medium stores further instructions that, when executed by the at least one processor, cause the system to train the structure-phenomics relationship neural network by:
generating a difference metric between the first phenomic embedding and the second phenomic embedding; and
applying a pheno-similarity threshold to the difference metric to generate a pheno-similarity classification between the training compound and the training perturbation,
wherein the pheno-similarity threshold is determined from a distribution of difference metrics between the training perturbation and a plurality of additional perturbations.

14. The system of claim 10, wherein the at least one non-transitory computer-readable storage medium stores further instructions that, when executed by the at least one processor, cause the system to train the structure-phenomics relationship neural network by:
modifying the parameters of the structure-phenomics relationship neural network to reduce a difference between the predicted phenomic feature space similarity and the phenomic image feature space similarity on a subsequent training iteration based on the phenomic feature space similarity measure of loss.

15. The system of claim 10, wherein the at least one non-transitory computer-readable storage medium stores additional instructions that, when executed by the at least one processor, cause the system to:
receive the query chemical compound and the target perturbation via a user interface of a client device; and
provide the phenomic similarity prediction for display via the user interface of the client device.

16. A non-transitory computer-readable medium storing instructions that, when executed by at least one processor, cause a computing device to:
train a structure-phenomics relationship neural network to generate phenomic similarity predictions by:
generating, utilizing a trained embedding neural network, a first phenomic embedding from a first phenomic image of a first cell exposed to a training compound;
generating, utilizing the trained embedding neural network, a second phenomic embedding from a second phenomic image of a second cell exposed to a training perturbation;
comparing, within a latent feature space of the trained embedding neural network, the first phenomic embedding and the second phenomic embedding to generate a phenomic image feature space similarity;
generating, utilizing the structure-phenomics relationship neural network, a predicted phenomic feature space similarity between the training compound and the training perturbation from a training compound structure feature representation of the training compound;
comparing the phenomic image feature space similarity and the predicted phenomic feature space similarity to generate a phenomic feature space similarity measure of loss; and
modifying parameters of the structure-phenomics relationship neural network utilizing the phenomic feature space similarity measure of loss;
generate a compound structure feature representation for a query chemical compound; and
generate, utilizing the structure-phenomics relationship neural network, a phenomic similarity prediction for the compound structure feature representation and a target perturbation.

17. The non-transitory computer-readable medium of claim 16, further storing additional instructions that, when executed by the at least one processor, cause the computing device to train the structure-phenomics relationship neural network by:
modifying parameters of a first task head of the structure-phenomics relationship neural network based on the predicted phenomic feature space similarity corresponding to the training perturbation; and
modifying parameters of a second task head of the structure-phenomics relationship neural network based on an additional predicted phenomic feature space similarity corresponding to an additional training perturbation.

18. The non-transitory computer-readable medium of claim 16, additionally storing further instructions that, when executed by the at least one processor, cause the computing device to train the structure-phenomics relationship neural network by:
generating a difference metric between the first phenomic embedding and the second phenomic embedding; and
applying a pheno-similarity threshold to the difference metric to generate a pheno-similarity classification between the training compound and the training perturbation, wherein the pheno-similarity threshold is determined from a distribution of difference metrics between the training perturbation and a plurality of additional perturbations.

19. The non-transitory computer-readable medium of claim 16, additionally storing further instructions that, when executed by the at least one processor, cause the computing device to train the structure-phenomics relationship neural network by:
modifying the parameters of the structure-phenomics relationship neural network to reduce a difference between the predicted phenomic feature space similarity and the phenomic image feature space similarity on a subsequent training iteration based on the phenomic feature space similarity measure of loss.

20. The non-transitory computer-readable medium of claim 16, further storing additional instructions that, when executed by the at least one processor, cause the computing device to:
receive the query chemical compound and the target perturbation via a user interface of a client device; and
provide the phenomic similarity prediction for display via the user interface of the client device.
